SEQUENCE LISTING (13 sequences)

SEQ ID NO: 1 - DNA, 15 bp, Artificial Sequence
  probe
gttagcggtaacgcg

SEQ ID NO: 2 - DNA, 15 bp, Artificial Sequence
  probe
gttagtggaagcgtc

SEQ ID NO: 3 - DNA, 20 bp, Artificial Sequence
  probe
gtgttagtggaagcgtctgg

SEQ ID NO: 4 - DNA, 25 bp, Artificial Sequence
  probe
atggggttagcggtaacgcgaagct

SEQ ID NO: 5 - DNA, 15 bp, Artificial Sequence
  probe
atgcacatattgtga

SEQ ID NO: 6 - DNA, 20 bp, Artificial Sequence
  probe
gtgctgaagcaacaaatgcc

SEQ ID NO: 7 - DNA, 25 bp, Artificial Sequence
  probe
cggtgctgaagcgacaaatgccctg

SEQ ID NO: 8 - DNA, 18 bp, Artificial Sequence
  primer
ttgtacacaccgcccgtc

SEQ ID NO: 9 - DNA, 23 bp, Artificial Sequence
  primer
tttcgcctttccctcacggtact

SEQ ID NO: 10 - DNA, 22 bp, Artificial Sequence
  primer
agtaccgtgagggaaaggcgaa

SEQ ID NO: 11 - DNA, 20 bp, Artificial Sequence
  primer
tgcttctaagccaacatcct

SEQ ID NO: 12 - DNA, 20 bp, Artificial Sequence
  primer
aggatgttggcttagaagca

SEQ ID NO: 13 - DNA, 24 bp, Artificial Sequence
  primer
cccgacaaggaatttcgctacctt

(19) United States
(12) Patent Application Publication
(10) Pub. No.: US 2010/0167956 A1
(43) Pub. Date: Jul. 1, 2010

(54) DNA CHIP FOR DETECTION OF ESCHERICHIA COLI

(75) Inventors: Sang Yup LEE, Daejeon (KR); Seung Min YOO, Seoul (KR); So Youn SHIN, Seoul (KR); Ki-Chang Keum, Seoul (KR); Nae-Choon YOO, Seoul (KR); Won-Min YOO, Seoul (KR); June-Myung KIM, Seoul (KR); Jun Yong CHOI, Seoul (KR)

Correspondence Address: MOORE & VAN ALLEN PLLC, P.O. BOX 13706, Research Triangle Park, NC, 27709, US

(73) Assignee: MEDIGENES CO., LTD., Seoul (KR)

(21) Appl. No.: 12/522,390

(22) PCT Filed: Jan. 8, 2007
(86) PCT No.: PCT/KR07/00123

§ 371 Date: Jan. 6, 2010

(51) Int. Cl. C40B 40/08; C07H 21/04

(52) U.S. Cl. 506/17; 536/24.32

(57) ABSTRACT

The present invention relates to nucleic acid probes specific to E. coli, which is useful for detecting and identifying E. coli in a biological sample. More particularly, the present invention relates to a DNA chip for detecting and identifying E. coli, on which nucleic acid probes derived from 23 S rRNA gene of E. coli are immobilized. The application of the DNA chip according to the present invention allows time-saving and accurate diagnosis of bacterial infection compared with the conventional of bacterial culture methods. In addition, it is not affected by antibiotics addition in clinical practice, thus leading to more accurate diagnosis.

### TECHNICAL FIELD

The present invention relates to nucleic acid probes specific to E. coli, which is useful for detecting and identifying E. coli in a biological sample. More particularly, the present invention relates to a DNA chip for detecting and identifying E. coli, on which nucleic acid probes derived from 23S rRNA gene of E. coli are immobilized.

### BACKGROUND ART

Infectious diseases, which are resulted from the presence and activity of pathogenic organisms in human blood, fluid, and tissue, may be developed into fatal diseases, if causal organisms fail to be identified and controlled properly. Recently, there has been abuse of antibiotic substances, overuse of immunosuppressants by transplantation and overdose of drugs by anticancer therapy. As results, pathogenic organisms are undergoing successive or alternate changes in genes and culture rate of such organisms is dwindling. The adaptation of pathogenic organisms makes it difficult to diagnose infectious diseases using traditional diagnostic methods. Since some anaerobic organisms exhibit enough pathogenicity to cause severe disease to humans, the rapid detection and accurate identification of pathogenic microbes in a biological sample are considerably of the importance in the treatment of infectious diseases.

E. coli, one of these infectious pathogenic microbes produces vero toxin to cause enterohemorrhagic E. coli infection, and so far, more than 70 E. coli serotypes are known to produce toxin. Enterohemorrhagic E. coli infection is food-borne disease mainly found in developed countries including USA and Japan, which causes a complication called hemolytic uremic syndrome and if it become severe, it could lead to death. In the USA, more than 20,000 cases of enterohemorrhagic E. coli infection, which resulted in 250 deaths, are estimated to occur each year (James et al, 1998). In Japan, around 100 cases thereof occurred until 1996, and now, each year 2000 cases are reported since a sharp increase to 3,022 cases in 1996. In Korea, enterohemorrhagic E. coli infection was designated in 2000 as a communicable disease (category I) under the communicable Disease Prevention Law, and less than 10 cases thereof occurred every year since one person was afflicted with the disease in 1998. Until 2002, a pandemic of enterohemorrhagic E. coli infection has not yet occurred, but it is predicted that it will become pandemic due to westernized diet.

Therefore, a variety of methods for the detection and identification of E. coli causing infectious diseases has been researched and developed over a long period of time. Although the technology for the detection of microbes has been remarkably advanced, it is still laborious and offers low sensitivity and specificity.

With the exception of viruses, all prokaryotic organisms contain rRNA genes encoding homologues of prokaryotic 5S, 16S and 23S rRNA molecules. In eukaryotic organisms, these rRNA molecules are 5S rRNA, 5.8S rRNA, 18S rRNA and 28S rRNA, which are substantially similar to the prokaryotic molecules.

Nucleic acid probes for detecting specifically targeted rRNA subsequences in particular non-viral organisms or groups of non-viral organisms in a biological sample have been described previously. Many of the problems in the conventional diagnostic methods could be solved by using such nucleic acid probes in combination with well-known polymerase chain reaction (PCR) techniques. The choice of target genes to be amplified is very important in a diagnostic nucleic acid probe technology and rRNA genes, especially 23S rRNA genes, are usually used as targeted sequences. It has been reported that nucleic acid probe sequences derived from rRNA genes advantageously allow low probability of cross-reacting with nucleic acids originating from microbes other than the targeted species under appropriate stringency conditions (P. Wattiau et al., Appl. Microbiol. Biotechnol., 56:816-819, 2001; D. A. Stahlm et al., J. Bacteriol., 172:116-124, 1990; Boddinghaus et al., J. Clin., Microbiol., 28:1751-1759, 1990; T. Rogall et al., J. Gen. Microbiol., 136:1915-1920, 1990; T. Rogall et al., Int. J. System. Bacteriol., 40:323-330, 1990; K. Rantakokko-Jalava et al., J. Clin., Mirobiol., 38(1):32-39, 2000; Park et al., J. Clin., Mirobiol., 38 (10:4080-4085, 2000; A. Schmalenberger et al., Appl. Microbiol. Biotechnol., 67(8):3557-3563, 2001; WO 98/55646; U.S. Pat. No. 6,025,132; and U.S. Pat. No. 6,277,577).

The present inventors have filed a patent (WO 03/095677A1) relating to a DNA chip using nucleic acid sequences specific to said infectious organisms as a probe in order to detect and identify non-viral infectious organisms, and have been studying to improve sensitivity of the DNA chip.

Accordingly, the present inventors have made extensive efforts to rapidly and accurately detect E. coli in samples from patients allegedly infected with E. coli. As a result, the present inventors have isolated distinct sequences specific to E. coli from 23S rRNA gene of E. coli genome, constructed a DNA chip using the sequences as probes for E. coli detection, and confirmed that E. coli can be detected in a rapid and accurate manner using the DNA chip, thereby completing the present invention.

### SUMMARY OF INVENTION

The main object of the present invention is to provide a DNA chip for detecting and identifying E. coli, on which oligonucleotides comprising sequences specific to E. coli, isolated from 23S rRNA gene of E. coli genome are fixed.

Another object of the present invention is to provide probes for detecting and identifying E. coli, which comprise the oligonucleotides.

To achieve the above object, the present invention provides probes for detecting and identifying E. coli, which comprise one or more oligonucleotides selected from the group consisting of oligonucleotides having nucleotide sequences of SEQ ID NOs: 1-7.

The present invention also provides a DNA chip for detecting and identifying E. coli, wherein said probes are fixed on a substrate.

In the present invention, the DNA chip preferably has all oligonucleotides having nucleotide sequences of SEQ ID NOs: 1-7 fixed thereon.

Other features and embodiments of the present invention will be more fully apparent from the following detailed description and appended claims.

### BRIEF DESCRIPTION OF DRAWINGS

FIG. 1A shows a schematic representation of the DNA chip designed for a blind test of a sample including E. coli. FIG. 1B shows the result of hybridization on the DNA chip in the blind test of the sample including E. coli, detected by ArrayWorks microarray scanner.

FIG. 2A shows a schematic representation of the DNA chip designed for a blind test of a sample including E. coli. FIG. 2B shows the hybridization result of Blind test using the sample including E. coli, detected by ArrayWorks microarray scanner.

### DETAILED DESCRIPTION OF THE INVENTION AND PREFERRED EMBODIMENTS

The present invention, in one aspect, relates to probes for detecting and identifying E. coli, which comprise oligonucleotides containing sequences specific to E. coli, derived from 23S rRNA gene of E. coli genome.

In the present invention, 23S rRNA nucleotide sequences of E. coli were compared with those of other microbes, whose sequences have been identified by performing multiple alignment, to determine sequences specific to E. coli, and candidate probes were selected using the sequences specific to E. coli, thus synthesizing probes specific to E. coli.

The present invention, in another aspect, relates to a DNA chip for detecting and identifying E. coli, on which oligonucleotides comprising sequences specific to E. coli, derived from 23S rRNA gene of E. coli genome are fixed.

In the present invention, the DNA chip for detecting E. coli was constructed by immobilizing said synthesized probes for detecting and identifying E. coli on a glass slide with an aldehyde-amine interaction.

Additionally, in order to verify the specificity of said probes and DNA chip for detecting E. coli, the genome of a standard strain was isolated to use as a template for PCR, and then the resulting PCR product was hybridized on the DNA chip, thus confirming that the DNA chip was effective in detecting E. coli. Moreover, contrary to the fact that E. coli detection rate of the conventional culture method was affected by antibiotic addition, it was confirmed through experimental results that the inventive DNA chip showed high detection efficacy even with antibiotic addition.

The following definitions serve to illustrate the terms and expressions used in the different embodiments of the present invention as set out below.

An “isolated” nucleic acid molecule is one separated from other nucleic acid molecules which are present in the natural source of the nucleic acid. For example, with regards to genomic DNA, the term “isolated” includes nucleic acid molecules separated from the chromosome with which the genomic DNA is naturally associated.

The term “probe” or “nucleic acid probe” refers to single stranded sequence-specific oligonucleotides which have a base sequence sufficiently complementary to hybridize to the target base sequence to be detected.

By “composition”, it is meant that probes complementary to bacterial or fungal rRNA may be in a pure state or in combination with other probes. In addition, the probes may be in combination with salts or buffers, and may be in a dried state, in an alcohol solution as a precipitate, or in an aqueous solution.

The term “target” refers to nucleic acid molecules originated from a biological sample which have a base sequence complementary to the nucleic acid probe of the invention. The target nucleic acid can be single- or double-stranded DNA (if appropriate, obtained following amplification) or RNA and contains a sequence which has at least partial complementarity with at least one probe oligonucleotide.

The phrase “a biological sample” refers to a specimen such as a clinical sample (pus, sputum, blood, urine, etc.), an environmental sample, bacterial colonies, contaminated or pure cultures, purified nucleic acid, etc. in which the target sequence of interest is sought.

By “oligonucleotide” is meant a nucleotide polymer generally consisting of about 10 to about 100 nucleotides in length, but which may be greater than 100 or shorter than 10 nucleotides in length.

By “nucleotide” is meant a subunit of a nucleic acid consisting of a phosphate group, a 5-carbon sugar and a nitrogen containing base. In RNA the 5-carbon sugar is ribose. In DNA, it is a 2-deoxyribose. For a 5-nucleotide, the sugar contains a hydroxyl group (—OH) at the carbon-5. The term also includes analogs of such subunits.

The term “homologous” is synonymous for identical and means that polynucleic acids which are said to be e.g. 90% homologous show 90% identical base pairs in the same position upon alignment of the sequences.

“Hybridization” involves the annealing of a complementary sequence to the target nucleic acid (the sequence to be detected). The ability of two polymers of nucleic acid containing complementary sequences to find each other and anneal through base pairing interaction is a well-recognized phenomenon.

The term “primer” refers to a single stranded DNA oligonucleotide sequence capable of acting as a point of initiation for synthesis of a primer, extension product which is complementary to the nucleic acid strand to be copied. The length and the sequence of the primer must be designed such that they allow to prime the synthesis of the extension products. Preferably the primer is about 5-50 nucleotides long. Specific length and sequence will depend on the complexity of the required DNA or RNA targets, as well as on the conditions of primer use such as temperature and ionic strength.

The term “label” as used herein refers to any atom or molecule which can be used to provide a detectable (preferably quantifiable) signal, which can be attached to a nucleic acid. Labels may provide signals detectable by fluorescence, radioactivity, colorimety, gravimety, X-ray diffraction or absorption, magnetism, and the like. By “hybrid” is meant the complex formed between two single stranded nucleic acid sequences by Watson-Crick base pairings or non-canonical base pairings between the complementary bases.

The phrase “probe specificity” refers to characteristic of a probe, which describes its ability to distinguish target and non-target sequences. In this regard, the term “specific” means that a nucleotide sequence will hybridize to a defined target sequence and will substantially not hybridize to a non-target sequence or that hybridization to a non-target sequence will be minimal. Probe specificity is dependent on sequence and assay conditions.

The phrase “standard strain” includes those commercially or readily available in the art.

### Identification of Probes

Each probe needs to be specific for the microbe of interest. The specific probes according to the present invention are designed as follows. First, specific nucleotide sequences solely present in the microbe of interest are identified by performing multiple alignment of nucleotide sequences derived from all possible microorganism species. The multiple alignment is carried out of 23S rRNA gene from bacteria and 18S rRNA gene from fungi. A lot of segments from 23S rRNA gene, and 18S rRNA are selected as candidate probes. Second, the specificity of the candidate probe is confirmed by comparison to public databases containing nucleotide sequences using the BLAST analyses well known to those skilled in the art to apply strains to the hybridization, thus selecting probes reacting with the microbe of interest as probes for identification. Third, the sensitivity of the candidate probe is assayed by applying it for clinical trials on a variety of biological samples.

The probe of the present invention include at least 15-mer oligonucleotide and are preferably 70%, 80%, 90% or more than 95% homologous to the exact complement of the target sequence to be detected. Those probes are about 50 nucleotides long. Of course, probes consisting of more than 50 nucleotides can be used. The nucleotides as used in the present invention may be ribonucleotides, deoxyribonucleotides and modified nucleotides such as inosine or nucleotides containing modified groups, which do not essentially alter their hybridization characteristics.

### Use of Probes

The probes of the invention can be used, for diagnostic purposes, in investigating the presence or the absence of a target nucleic acid in a biological sample, according to all the known hybridization techniques and especially the techniques of point deposition on filter called “DOT-BLOT” (Maniatis et al., Molecular Cloning, Cold Spring Harbor, 1982), the DNA transfer techniques called “SOUTHERN BLOT” (Southern, E. M., J. Mol. Biol. 98:503, 1975)), or the RNA transfer techniques called “NORTHERN BLOT”.

The probes of the invention can also be used in a sandwich hybridization system which enhances the specificity of a nucleic acid probe-based assay. The principle and the use of sandwich hybridizations in a nucleic acid probe-based assay have been already described (e.g.: Dunn and Hassel, Cell, 12:23-36; 1977; Ranki et al., Gene, 21:77-85; 1983). The sandwich hybridization technique uses a capture probe and/or a detection probe, said probes being capable of hybridizing with two different regions of the target nucleic acid, and at least one of said probes (generally the detection probe) being capable of hybridizing with a region of the target which is specific for the species or the group of species investigated. It is understood that the capture probe and the detection probe must have nucleotide sequences which are at least partly different. Although direct hybridization assays have favorable kinetics, sandwich hybridizations are advantageous with respect to a higher signal-to-noise ratio. Moreover, sandwich hybridizations can enhance the specificity of a nucleic acid probe based assay. The incubation and subsequent washing stages which constitute the key stages of the sandwich hybridization process are each carried out at a constant temperature, between about 20° C. and 65° C. It is known that nucleic acid hybrids have a dissociation temperature which depends on the number of hybridized bases (the temperature increasing with the size of the hybrid) and which also depends on the nature of the hybridized bases and, for each hybridized base, depends on the nature of the adjacent bases. The hybridization temperature used in the sandwich hybridization technique should obviously be chosen below the half-dissociation temperature of the hybrid formed between a given probe and the target of complementary sequence, by simple routine experiment.

The probes of the present invention can also be used in a competition hybridization protocol. In a competition hybridization, the target molecule competes with the hybrid formation between a specific probe and its complement. The more target is present, the lower the amount of hybrid formed between the probe and its complement is. A positive signal, which indicates that the specific target was present, is seen by a decrease in hybridization reaction as compared with a system to which no target was added. In a particular embodiment, the specific oligonucleotide probe, conveniently labeled, is hybridized with the target molecule. Next, the mixture is transferred to a recipient (e.g. a microtiter dish well) in which an oligonucleotide complementary to the specific probe is fixed and the hybridization is continued. After washing, the hybrids between the complementary oligonucleotide and the probe are measured, preferably quantitatively, according to the label used.

In addition, the probes of the present invention can be used in a reversed hybridization (Proc. Natl. Acad. Sci. USA, 86:6230-6234, 1989). In this case, the target sequences can first be enzymatically amplified by performing PCR with 5′ biotinylated primers. In a second step, the amplified products are detected upon hybridization with specific oligonucleotides immobilized on a solid support. Reversed hybridization may also be carried out without an amplification step. In that particular case, the nucleic acids present in the sample have to be labeled or modified, specifically or not, for instance, chemically or by addition of specific dyes, prior to hybridization.

The nucleic acid probes of the present invention can be included in a kit which can be used to rapidly determine the presence or absence of pathogenic species of interest. The kit includes all components necessary to assay for the presence of these pathogens. In the universal concept, the kit includes a stable preparation of labeled probes, hybridization solution in either dry or liquid form for the hybridization of target and probe polynucleotides, as well as a solution for washing and removing undesirable and nonduplexed polynucleotides, a substrate for detecting the labeled duplex, and optionally an instrument for the detection of the label.

A more specific embodiment of the present invention embraces a kit that utilizes the concept of the sandwich assay. This kit would include a first component for the collection of samples from patients, such as a scraping device or paper points, vials for containment, and buffers for the dispersement and lysis of the sample. A second component would include media in either dry or liquid form for the hybridization of target and probe polynucleotides, as well as for the removal of undesirable and nonduplexed forms by washing. A third component includes a solid support on which is fixed or to which is conjugated unlabeled nucleic acid probe(s) that is (are) complementary to a part of the target polynucleotide. In the case of multiple target analysis, more than one capture probe, each specific for its own ribosomal RNA, will be applied to different discrete regions of the dipstick. A fourth component would contain a labeled probe that is complementary to a second and different region of the same rRNA strand to which the immobilized, unlabeled nucleic acid probe of the third component is hybridized. The probe components described herein include combinations of probes in dry form, such as lyophylized nucleic acid or in precipitated form, such as alcohol precipitated nucleic acid or in buffered solutions. The label may be any of the labels described above. For example, the probe can be biotinylated using conventional means and the presence of a biotinylated probe can be detected by adding avidin conjugated to an enzyme, such as horseradish peroxidase to contact with a substrate which, when reacted with peroxidase, can be monitored visually or by instrumentation using a colorimeter or spectrophotometer. This labeling method and other enzyme-type labels have advantages of being economical, highly sensitive, and relatively safe compared to radioactive labeling methods. Various reagents for the detection of labeled probes and other miscellaneous materials for the kit, such as instructions, positive and negative controls, and containers for conducting mixing, and reacting various components, would complete the assay kit.

### DNA Chip

The probes of the present invention are also used in a DNA chip. In a preferred embodiment, the present invention provides a DNA chip in which nucleic acid probes are immobilized on a solid support. The DNA chip which is formed by arranging DNA fragments of variety of base sequences on the surface of a narrow substrate in high density is used in finding out the information on DNA of an unknown sample by hybridization between immobilized DNA and unknown DNA sample complementary thereto. Examples of the solid carrier on which the probe oligonucleotides are fixed include inorganic materials such as glass and silicon and polymeric materials such as acryl, polyethylene terephtalate (PET), polystyrene, polycarbonate and polypropylene. The surface of the solid substrate can be flat or have multiple holes. The probes are immobilized on the substrate by covalent bond of either 3′ end or 5′ end. The immobilization can be achieved by conventional techniques, for example, using electrostatic force, binding between aldehyde coated slide and amine group attached on synthetic ologomeric phase or spotting on amine coated slide, L-lysine coated slide or nitrocellulose coated slide. One embodiment of the present invention includes incorporating base with amino residue on 3′ position of the probe upon synthesizing it, followed by covalently binding it on aldehyde coated glass slide.

The immobilization and the arrangement of various probes onto the solid substrate are carried out by pin microarray, inkjet, photolithography, electric array, etc. In an embodiment of the invention, probes are separately dissolved in a buffer solution and the resulting solution is spotted onto the substrate by using a microarrayer prepared by a known method (Yoon et al., J. Microbiol. Biotechnol., 10(1):21-26, 2000). The basis principle of the microarrayer is that minutely constructed pin picks probe DNAs from a plate and transfers it to the site that is appointed by a computer. For the fixing of the probe transferred by a microarrayer, the immobilization reaction is allowed for at least one hour under humidity of from 45% to 65%, preferably, from 50% to 55%, and it stands up for at least 6 hours to facilitate the reaction between the amine group at 3′ position of the probe and the aldehyde group coated onto the glass slide.

For detecting cells derived from or themselves being living organisms, the RNA and/or DNA of these cells, if necessary, is made accessible by partial or total lysis of the cells using chemical and/or physical processes, and contacted with one or several probes of the present invention which can be detected. This contact can be carried out on an appropriate support such as a nitrocellulose, cellulose, or nylon filter in a liquid medium or in solution.

This contact can take place under suboptimal, optimal conditions, or under restrictive conditions. Such conditions include temperature, concentration of reactants, the presence of substances lowering the optimal temperature of pairing of nucleic acids (e.g. formamide, dimethylsulfoxide and urea) and the presence of substances apparently lowering the reaction volume and/or accelerating hybrid formation (e.g. dextran sulfate, polyethyleneglycol or phenol).

### Preparation of Probes

To obtain large quantities of nucleic acid probes, one can either clone the desired sequence using traditional cloning methods, such as described in Maniatis, T., et al. Molecular Cloning: A Laboratory Manual, Cold Spring Harbor, N.Y., 1982, or one can produce the probes by chemical synthesis using commercially available DNA synthesizers.

The probes of the present invention can be prepared by conventional methods. Two methods are typically introduced. A first method is a preparation of a single-stranded probe. A representive example of preparing a single-stranded probe consisting of the desired number of nucleotides includes a dimethoxytrityl (DMT) off method by an automated DNA synthesizer which comprises removing the DMT group to free the 5′ hydroxyl for the coupling reaction, coupling and capping. The probes obtained thereby is labeled with a fluorescent dye (fluorescein isothiocyanate, FITC) to confirm the presence or the absence of nucleic acids of interest. Alternatively, the DNA probe complementary to single-stranded DNA template is prepared by annealing the primer to the template DNA and performing extension reactions from the primer/template complex using Klenow fragment and dNTP labeled with fluorescent dye. The probe made thus exhibits high sensitivity and specificity owing to its fluorescent dye.

A second method is a preparation of double-stranded probe. It is possible to make a probe having the desired region of a gene or a base segment by digesting genomic DNA or plasmid DNA with specific restriction enzymes. A random priming method is a synthesis of fluorescent-labeled probes with various lengths by hybridizing six random hexamer with template DNA. Alternatively, fluorescent-labeled probes can be synthesized by transferring ³²P to the 5′ end of DNA by T4 polynucleotide kinase. In addition, the probe can be synthesized by breaking down double-stranded DNA molecules with DNase I and performing DNA replication using DNA polymerase I and fluorescent-labeled dNTP. The double-stranded probe obtained thereby is denatured to form single-stranded DNAs which are then used in a hybridization reaction.

The probes of the present invention are advantageously labeled. Any conventional label can be used. The probes can be labeled by means of radioactive tracers such as ³²P, ³⁵S, ¹²⁵I, ³H and ¹⁴C. The radioactive labeling can be carried out according to any conventional method such as terminal labeling at the 3′ or 5′ position with the use of a radiolabeled nucleotide, a polynucleotide kinase (with or without dephosphorylation by a phosphatase), a terminal transferase, or a ligase. Another method for radioactive labeling is a chemical iodination of the probes of the present invention, which leads to the binding of several ¹²⁵I atoms on the probes.

If one of the probes of the present invention is made radioactive to be used for hybridization with a nonradioactive RNA or DNA, the method of detecting hybridization will depend on the radioactive tracer used. Generally, autoradiography, liquid scintillation, gamma counting or any other conventional method enabling one to detect an ionizing ray issued by the radioactive tracer can be used. Nonradioactive labeling can also be used by associating the probes of the present invention with residues having: immunological properties (e.g. antigen or hapten), a specific affinity for some reagents (e.g. ligand), properties providing a detectable enzymatic reaction (e.g. enzyme, co-enzyme, enzyme substrate or substrate taking part in an enzymatic reaction), or physical properties such as fluorescence, emission or absorption of light at any wavelength. Antibodies which specifically detect the hybrids formed by the probe and the target can also be used.

A nonradioactive label can be provided when chemically synthesizing a probe of the present invention and the adenosine, guanosine, cytidine, thymidine and uracyl residues thereof being liable to be coupled to other chemical residues enable the detection of the probe or the hybrids formed between the probe and a complementary DNA or RNA fragment.

### Target

To provide nucleic acid substrates for use in the detection and identification of microorganisms in clinical samples using the structure probing assay, nucleic acid is extracted from the sample. The nucleic acid may be extracted from a variety of clinical samples using a variety of standard techniques or commercially available kits. For example, kits which allow the isolation of RNA or DNA from tissue samples are available from Qiagen, Inc. (Chatsworth, Calif.) and Stratagene (La Jolla, Calif.). For example, the QIAamp Blood kits permit the isolation of DNA from blood (fresh, frozen or dried) as well as bone marrow, body fluids or cell suspensions. QIAamp tissue kits permit the isolation of DNA from tissues such as muscles, organs and tumors.

In a preferred method of determining whether a biological sample contains rRNA or rDNA that would indicate the presence of the desired pathogens, nucleic acids may be released from cells by sonic disruption, for example according to the method disclosed by Murphy et al., in U.S. Pat. No. 5,374,522. Other known methods for disrupting cells include the use of enzymes, osmotic shock, chemical treatment, and vortexing with glass beads. Other methods, suitable for liberating from microorganisms the nucleic acids that can be subjected to the hybridization disclosed herein, have been described by Clark et al., in U.S. Pat. No. 5,837,452 and by Kacian et al., in U.S. Pat. No. 5,364,763.

Following or concurrent with the release of rRNA, labeled probe may be added in the presence of accelerating agents and incubated at the optimal hybridization temperature for a period of time necessary to achieve significant hybridization reaction. In the case of a double-stranded nucleic target, it is advisable to carry out its denaturation before carrying out the process of detection. The denaturation of a double-stranded nucleic acid may be carried out by known methods of chemical, physical or enzymatic denaturation, and in particular by heating at an appropriate temperature, higher than 80° C.

In addition, target DNA hybridizing to the probe is usually prepared by two methods. A first method is one used in Southern blot or Northern blot. Genomic DNAs or plasmid DNAs are digested with appropriate restriction enzymes and the resulting DNA fragments are separated by agarose gel electrophoresis and used. A second method is an amplification of the desired DNA region by PCR. Examples of the PCR include most typical PCR using the same amounts of forward and reverse primers, asymmetric PCR in which double-stranded and single-stranded bands can be obtained by adding primers asymmetrically, multiplex PCR in which a multiple of target DNAs can be amplified at once by adding various primers simultaneously, ligase chain reaction (LCR) in which target DNA is amplified using specific 4 primers and ligase and the amount of fluorescence is measured by ELISA (Enzyme Linked Immunosorbent Assay), and the other PCR such as Hot Start PCR, Nest-PCR, DOP-PCR (degenerate oligonucleotide primer PCR), RT-PCR (reverse transcription PCR), Semi-quantitative RT-PCR, Real time PCR, RACE (rapid amplification of cDNA ends), Competitive PCR, STR (short tandem repeats), SSCP (single strand conformation polymorphism), DDRT-PCR (differential display reverse transcriptase), etc.

It has been found that crude extracts from relatively homogenous specimens (such as blood, bacterial colonies, viral plaques, or cerebral spinal fluid) are better suited to serve as templates for the amplification of unique PCR products than are more composite specimens (such as urine, sputum or feces) (Mullis et al., Shibata in PCR: The Polymerase Chain Reaction, eds., Birkhauser, Boston, pp. 47-54, 1994,). Samples which contain relatively few copies of the material to be amplified (i.e., the target nucleic acid), such as cerebral spinal fluid, can be added directly to a PCR. Blood samples have posed a special problem in PCRs due to the inhibitory properties of red blood cells. The red blood cells must be removed prior to the use of blood in a PCR; there are both classical and commercially available methods for this purpose (e.g., QIAamp Blood kits, passage through a Chelex 100 column [BioRad], etc.). Extraction of nucleic acid from sputum, the specimen of choice for the direct detection of M. tuberculosis, requires prior decontamination to kill or inhibit the growth of other bacterial species. This decontamination is typically accomplished by treatment of the sample with N-acetyl L-cysteine and NaOH (Shinnick and Jones, supra). This decontamination process is necessary only when the sputum specimen is to be cultured prior to analysis.

A preferred embodiment of the present invention includes preparing gene fragments by an asymmetric PCR using DNA of isolated sample as a template. The gene fragments are obtained by performing the PCR at once with addition of forward and reverse primers at the ratio of 1:5.

The used primers correspond to the regions of 16S rRNA or 23S rRNA universally present in bacteria (Pirkko K. et al., Clin. Microbiol., 36 (8), 2205-2209, 1999) and are as follows:

| | |
|---|---|
| Primer 1-S (sense): | |
| P-TTGTACACACCGCCCGTC, | (SEQ ID NO: 8, |
| | 1585Fw) |
| Primer 1-A (antisense): | |
| Cy3-TTTCGCCTTTCCCTCACGGTACT, | (SEQ ID NO: 9, |
| | 23Br) |
| Primer 2-S (sense): | |
| P-AGTACCGTGAGGGAAAGGGGAA, | (SEQ ID NO: 10, |
| | 23BFw) |
| Primer 2-A (antisense): | |
| Cy3-TGCTTCTAAGCCAACATCCT, | (SEQ ID NO: 11, |
| | MS37R) |
| Primer 3-S (sense): | |
| P-AGGATGTTGGCTTAGAAGCA, | (SEQ ID NO: 12, |
| | MS37F) |
| Primer 3-A (antisense): | |
| Cy3-CCCGACAAGGAATTTCGCTACCTT. | (SEQ ID NO: 13, |
| | MS38R) |

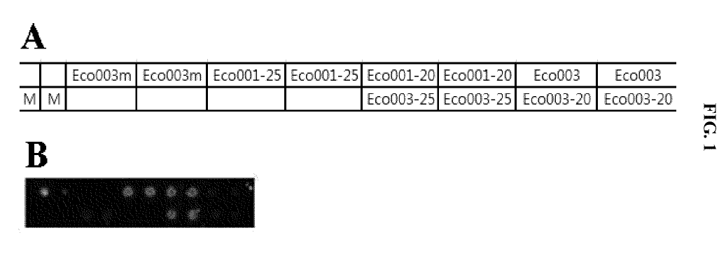

In the above primers, the locations are shown in FIG. 1 and the letter “F” conjugated to 5′ end indicates fluorescein isothicyanate (FITC). The target DNAs are amplified using 5-FITC conjugated primers, and then the hybridization between the amplified target DNAs and the nucleic acid probes is determined by fluorescence to confirm the identity of the infectious agent. In order to obtain the regions which cannot be amplified by the above primers, additional primers are designed through multiple alignment and BLAST.

In a preferred embodiment of the PCR, 5 μl of 10×PCR buffer solution (100 mM Tris-HCl, pH 8.3, 500 mM KCl, 15 mM MgCl₂), 4 μl of dNTP mixture (dATP, dGTP, dCTP, dTTP, each 2.5 mM), 0.5 μl of 10 pmole forward primer, 2.5 μl of 10 pmole reverse primer, 1 μl of 1/10 diluted template DNA (100 ng) and 0.5 μl of Taq polymerase (5 unit/μl, Takara Shuzo Co., Shiga, Japan) are mixed and water is added to the resulting mixture to be a total volume of 50 μl. The asymmetric PCR is conducted by 10 cycles, each consisting of first denaturation at 94° C. for 7 minutes, second denaturation at 94° C. for 1 minute, annealing at 52° C. for 1 minute and extension at 72° C. for 1 minute, and 30 cycles, each consisting of third denaturation at 94° C. for 1 minute, annealing at 54° C. for 1 minute and extension at 72° C. for 1 minute, followed by one final extension at 72° C. for 5 minutes. The PCR products are confirmed by agarose gel electrophoresis.

### Hybridization and Wash

A particular hybridization technique is not essential to the present invention. Hybridization techniques are generally described in prior art (Gall and Pardue, Proc. Natl. Acad. Sci., U.S.A, 63:378-383, 1969; and John et al., Nature, 223:582-587, 1969).

The hybridization conditions are determined by the “stringency”, that is to say the strictness of the operating conditions. The hybridization becomes more specific when it is carried out with greater stringency. The stringency is a function especially of the base composition of a probe/target duplex, as well as by the degree of mismatching between two nucleic acids. The stringency can likewise be a function of parameters of the hybridization reaction, such as the concentration and the type of ionic species present in the hybridization solution, the nature and the concentration of denaturing agents and/or hybridization temperature. The stringency of the conditions under which a hybridization reaction must be carried out depends especially on the probes used. All these data are well known and the appropriate conditions can possibly be determined in each case by routine experiments. In general, depending on the length of the probes used, the temperature for the hybridization reaction is between approximately 20° C. and 65° C. in particular between 35° C. and 65° C., in a saline solution at a concentration of approximately 0.8 to 1 M.

Nucleic acid hybridization between labeled oligonucleotide probes and nucleic acid targets can be enhanced by the use of “unlabeled Helper Probes” as disclosed in U.S. Pat. No. 5,030,557 to Hogan et al. Helper probes are oligonucleotides which bind to a portion of the target nucleic acid other than that being targeted by the assay probe, and which imposes new secondary and tertiary structure on the targeted region of the single stranded nucleic acid whereby the rate of binding of the assay probe is accelerated.

It will be appreciated by those skilled in the art that factors, which affect the thermal stability, can also affect probe specificity and therefore, must be controlled. Thus, the melting profile, including the melting temperature (Tm) of the oligonucleotide/target hybrids should be determined. The preferred method is described in U.S. Pat. No. 5,283,174. For Tm measurement using a Hybridization Protection Assay, the following technique is used. A probe:target hybrid is formed in target excess in a lithium succinate buffered solution containing lithium lauryl sulfate. Aliquots of this “preformed” hybrid are diluted in the hybridization buffer and incubated for five minutes at various temperatures starting below the anticipated Tm (typically 55° C.) and increasing in 2˜5° C. increments.

This solution is then diluted with a mildly alkaline borate buffer and incubated at a lower temperature (for example 50° C.) for ten minutes. Under these conditions, the acridinium ester attached to a single stranded probe is hydrolyzed while that attached to hybridized probe is relatively “protected”. This is referred to as the hybridization protection assay (“HPA”). The amount of chemiluminescence remaining is proportional to the amount of hybrid and is measured in a luminometer by addition of hydrogen peroxide followed by alkali. The data is plotted as percent of maximum signal (usually from the lowest temperature) versus temperature. The Tm is defined as the point at which 50% of the maximum signal remains.

In addition to the above method, oligonucleotide/target hybrid melting temperature may also be determined by isotopic methods well known to those skilled in the art. It should be noted that the Tm for a given hybrid will vary depending on the hybridization solution being used because the thermal stability thereof depends upon the concentration of different salts, detergents, and other solutes which affect relative hybrid stability during thermal denaturation. (Sambrook et al., Molecular Cloning: A Laboratory Manual, eds. Cold Spring Harbor Lab Publ., 9.51 (2nd ed.), 1989).

The hybridization conditions can be monitored relying upon several parameters, e.g. hybridization temperature, the nature and concentration of the components of the media, and the temperature under which the hybrids formed are washed. The hybridization and wash temperature is limited to upper value, according to the probe (its nucleic acid composition, kind and length) and the maximum hybridization or wash temperature of the probes described herein is about 30° C. to 60° C. At higher temperatures, the duplexing competes with the dissociation (or denaturation) of the hybrid formed between the probe and the target. A preferred hybridization medium contains about 3×SSC (1×SSC=0.15 M NaCl, 0.015 M sodium citrate, pH 7.0), about 25 mM of phosphate buffer pH 7.1, and 20% deionized formamide, 0.02% Ficoll, 0.02% bovine serum albumin, 0.02% polyvinylpyrrolidone and about 0.1 mg/ml sheared denatured salmon sperm DNA. A preferred wash medium contains about 3×SSC, 25 mM phosphate buffer pH 7.1 and 20% deionized formamide. However, when modifications are introduced, to the probes and the media, the temperatures at which the probes can be used to obtain the required specificity should be changed according to the known relationships. In this respect, it should also be noted that, in general, DNA: DNA hybrids are less stable than RNA: DNA or RNA: RNA hybrids. Depending on the nature of the hybrid to be detected, the hybridization conditions should be adapted accordingly to achieve specific detection.

In a preferred embodiment of the present invention, a hybridization buffer solution (6×SSPE (0.15M NaCl, 5 mM C₆H₅Na₃0₇, pH 7.0), 20% (v/v) formamide) is mixed with PCR amplified target genes, the resulting mixture is applied onto a glass slide to which probes are immobilized, and then the reaction is kept at 30° C. for 6 hours so that the said probes can complementarily hybridize with the said targets. The glass slide is washed sequentially with 3×SPE, 2×SSPE and 1×SSPE for 5 minutes each.

The formed hybrids can be quantified by labeling the target with a fluorescence or radioactive isotope in accordance to the conventional methods. The labeling may be carried out by the use of labeled primers or the use of labeled nucleotides incorporated during the polymerase step of the amplification.

E. coli does not usually turn pathogenic in the intestine but, in the organs other than the intestine, the microbe provokes cystitis, pyelitis, peritonitis and sepsis. Moreover, antigenic types (serotypes) of E. coli including O-26, O-55 and O-111 occasionally cause infectious diarrhea in infants and adults, which is therefore dubbed pathogenic E. coli.

The inventive probes for detecting and identifying E. coli are applied to a DNA chip to detect E. coli with excellent specificity, and thus, can precisely diagnose infectious diseases caused by E. coli. In addition, the probes of the present invention can be used in combination of two or more species to a DNA chip for the simultaneous detection of multiple pathogen species possibly present in a particular type of a biological sample.

### EXAMPLES

Hereinafter, the present invention will be described in more detail by the following examples. However, it will be obvious to a person skilled in the art that these examples are given to provide a better understanding of the present invention and are not construed to limit the scope of the present invention.

### Example 1

### Selection of Candidate DNA Probes for Identifying E. Coli

In order to detect and identify E. coli, probes specific to E. coli were constructed. For the construction of specific probes, candidate probes specific to E. coli were selected. 23S rRNA sequence of E. coli listed in Genebank was compared to those of known microbes by a multiple alignment to determine specific sequences present only in E. coli, thus constructing candidate probes. The candidate probes were selected within 23S rRNA gene of E. coli. The specificity of candidate probes was confirmed by comparing sequence similarity between microbes using BLAST analysis. As a result, the candidate probes screened thereby are shown in Table 1 below.

TABLE 1
Candidate probes specific to E. coli

| | | | SEQ |
|---|---|---|---|
| Probe | | | ID |
| No. | Sequence | Nomenclature | NO: |
| 1 | GTTAGCGGTAACGCG | Eco001 | 1 |
| 2 | GTTAGTGGAAGCGTC | Eco001m | 2 |
| 3 | GTGTTAGTGGAAGCGTCTGG | Eco001-20 | 3 |
| 4 | ATGGGGTTAGCGGTAACGCGAAGCT | Eco001-25 | 4 |
| 5 | ATGCACATATTGTGA | Eco002 | 5 |
| 6 | GTGCTGAAGCAACAAATGCC | Eco003-20 | 6 |
| 7 | CGGTGCTGAAGCGACAAATGCCCTG | Eco003-25 | 7 |

### Example 2

### Synthesis of Nucleic Acid Probes

For the construction of DNA chip, candidate probes screened in the above Example 1 were chemically synthesized. Mononucleotides (Proligo Biochemie GmbH Hamburg Co.) were introduced into an Expedite 8900 nucleic acid synthesis system (PE Biosystems Co.) with input of the desired nucleotide sequence and scale to afford 0.05 μmole of pure nucleic acid probes. The resulting probes were confirmed by an electrophoresis.

### Example 3

### Construction of DNA Chip

In order to immobilize DNA probes on a solid support, amine-aldyhyde covalent bonds were used. The 3′ termini of synthetic oligonucleotide probes were modified with amine residues using an amino linker column (Cruachem, Glasgow, Scotland) for the immobilization on a glass slide. For the glass slide, the aldehyde-coated glass slide (CEL Associates, Huston, Tex.) was used. The probes were dissolved in 3×SSC (0.45M NaCl, 15 mM C₆H₅Na₃O₇, pH 7.0) spotting solution. The resulting solution was spotted on the slide glass surface using a microarrayer (MicroGrid Spotter, Biorobotics Inc, England). The slide glass was kept under about 55% humidity for 1 hour and then air-dried for 6 hours so that the DNA probes could be immobilized on the glass slide. All probes were spotted with intervals of 250˜275 μm at the concentration of 100 pmole. To evaluate immobilization efficiency, the glass slide was dyed with SYBRO green II (Molecular Probe, Inc., Leiden, Netherlands).

### Example 4

### Isolation and Amplification of Target DNA

Genomic DNAs were extracted from 59 standard strains set forth in Table 2 below.

TABLE 2
Source of 59 standard stains

| | Gram | | | Gram | |
|---|---|---|---|---|---|
| Species | Staining | Source | Species | Staining | Source |
| Neisseria | − | ATCC10150 | Acinetobacter | − | KCTC2771 |
| gonorrhoeae | | | baumannii | | |
| Neisseria | − | ATCC13100 | Eikenella corodens | − | ATCC51724 |
| meningitidis | | | | | |
| Legionella | − | Clinical | Actinomyces israeli | − | ATCC12102 |
| pneumophilia | | isolation | | | |
| Listeria | + | ATCC700603 | Anaerobiospirilum | − | ATCC29305 |
| monocytogenes | | | succiniciproducens | | |
| Morganella | − | ATCC25830 | Aeromonas hydrophila | − | KCCM32586 |
| morganii | | | | | |
| Bacteroid vulgatus | − | KCCM11423 | Escherichia coli | − | ATCC25922 |
| | | KCCM8482 | | | |
| Bacteroid ovatus | − | ATCC8483 | Enterobacter aerogenes | − | KCCM11783 |
| | | | | | ATCC29751 |
| Bacteroides | − | ACTC5015 | Enterobacter cloacae | − | KCCM40044 |
| thetaiotaomicron | | | | | |
| Bacteroides fragils | − | ATCC25282 | Enterococcus faecalis | + | ATCC19433 |
| Burkholderia | − | ATCC25416 | Enterococcus faecium | + | ATCC19434 |
| cepacia | | | | | |
| Branhamella | − | KCCM4006 | Ocrobactrum Anthropi | − | ATCC49188 |
| catarrhalis | | ATCC43617 | | | |
| Vibrio vulnifucus | − | KCTC2962 | Cardiobacterium | − | ATCC14900 |
| | | | hominis | | |
| Vibrio cholerae | − | KCTC2715 | Corinaebacterium | + | ATCC51696 |
| | | | diphteriae | | |
| Salmonella | − | KCCM12021 | Comamonas | − | ATCC9355 |
| enteritidis | | | acidovorans | | |
| Salmonella | − | KCCM40253 | Klebsiella pneumoniae | − | AATCC700603 |
| typhimurium | | | | | |
| Serratia marcescens | − | KCTC1299 | Klebsiella oxytoca | − | ATCC43863 |
| Sutterella | − | ATCC51579 | Chryseobacterium | − | ATCC13253 |
| wadsworthensis | | | meningosepticum | | |
| Shigella sonnei | − | KCCM11903 | Clostridium difficile | + | ATCC9689 |
| Shigella flexneri | − | ATCC11836 | Clostridium ramosum | + | ATCC25582 |
| Pseudomonas | − | KCTC1636 | Kingella kingae | − | ATCC23330 |
| aeruginosa | | | | | |
| Staphylococcus | + | KCTC1621 | Peptostreptococcus | + | ATCC29328 |
| aureus | | | magnus | | |
| Staphylococcus | + | KCTC1917 | Peptostreptococcus | + | ATCC27337 |
| epidermidis | | | anaerobius | | |
| Stomatococcus | + | ATCC17931 | Peptostreptococcus | + | KCTC3319 |
| mucilaginosus | | | prevotii | | |
| Sterntrophomonas | − | ATCC13637 | Phorphyromonas | + | ATCC33277 |
| maltophila | | | gingivalis | | |
| Streptococcus | + | KCCM11823 | Fusobacterium | − | ATCC25286 |
| mutans | | ATCC25175 | necrophorum | | |
| Streptococcus | + | ATCC35037 | Proteus mirabilis | − | KCCM11381 |
| viridans | | | | | |
| Streptococcus | + | KCCM11957 | Proteus vulgaris | − | KCCM11539 |
| agalactiae | | | | | |
| Streptococcus | + | KCCM11817 | Haemophilus | − | ATCC13252 |
| pyogenes | | | aprophilus | | |
| Streptococcus | + | KCCM40410 | Haemophylus influenzae | − | ATCC51907 |
| pneumoniae | | | | | |
| Citrobacter freundii | − | ATCC51579 | | | |

The microbial species was grown on a suitable medium and suspended in 200 μl of sterilized distilled water. The suspension was centrifuged at 14,000 rpm for 10 minutes. The supernatant was discarded to obtain a pellet.

For gram-negative species, the pellet was put into 180 μl of ATL solution (Tissue Lysis Solution, DNeasy Tissue Kit, QIAGEN). 20 μl of proteinase K was added to the solution to lyse cells. The resulting lysate was cultured at 55° C. for 1 hour. The culture was vortexed for 15 seconds and mixed with 200 μl of AL solution (Lysis Solution, DNeasy Tissue Kit, QIAGEN). The resulting mixture was cultured at 70° C. for 10 minutes. The culture was mixed with 200 μl of ethanol (100%). The resulting solution was loaded onto the DNeasy mini column sitting in a 2 ml tube and centrifuged at 8,000 rpm or more for 1 minute. The solution collected in the tube was discarded. 500 μl of AW1 solution (Wash Solution 1, DNeasy Tissue Kit, Qiagen) was pipetted into the column which was then centrifuged at 8,000 rpm for 1 minute. The elute was discarded and 500 μl of AW2 solution (Wash Solution 2, DNeasy Tissue Kit, Qiagen) was again pipetted into the column which was then centrifuged 1,500 rpm for 3 minutes. The DNeasy membrane was dried and the elute was discarded. The dry DNeasy mini column was placed in the tube and stood at room temperature for 15 minutes, and then centrifuged at 8,000 rpm for 1 minute to elute genomic DNAs.

For gram-positive species, the pellet was suspended into 180 μl of lysozyme solution (20 mM Tris-Cl, pH 8.0, 2 mM EDTA, 1.2% Triton X-100, 20 mg/ml lysozyme) and cultured at 37° C. for 30 minutes. The culture obtained thereby was mixed with 25 μl of proteinase K and 200 μl of AL solution (Lysis Solution, DNeasy Tissue Kit, QIAGEN). The resulting mixture was cultured at 70° C. for 30 minutes. And the ensuing steps of isolating genomic DNAs were performed in the same manner as described above.

PCR was carried out using DNAs isolated from standard strains as descried above as a template with primers below. DNA binding to probes upon PCR was examined by amplifying using reverse primers having Cy3 linked at the 5′ terminal end thereof, which is labeled with fluorescence for detection. Three pairs of primers below were simultaneously used for PCR reaction:

| | |
|---|---|
| Primer 1-S (sense): | |
| P-TTGTACACACCGCCCGTC | (SEQ ID NO: 8, |
| | 1585Fw) |
| Primer 1-A (antisense): | |
| Cy3-TTTCGCCTTTCCCTCACGGTACT; | (SEQ ID NO: 9, |
| | 23BR) |
| Primer 2-S (sense): | |
| P-AGTACCGTGAGGGAAAGGCGAA | (SEQ ID NO: 10, |
| | 23BFw) |
| Primer 2-A (antisense): | |
| Cy3-TGCTTCTAAGCCAACATCCT; | (SEQ ID NO: 11, |
| | MS37R) |
| Primer 3-S (sense): | |
| P-AGGATGTTGGCTTAGAAGCA | (SEQ ID NO: 12, |
| | MS37F) |
| and | |
| Primer 3-A (antisense): | |
| Cy3-CCCGACAAGGAATTTCGCTACCTT | (SEQ ID NO: 13, |
| | MS38R) |

“P” at 5′-end of the above sequences indicates a phosphate group and “Cy3” represents fluorescent substance Cy3.

PCR reaction were performed as follows: the PCR mixture containing 5 μl of 10×PCR buffer (100 mM Tris-HCl (pH 8.3), 500 mM KCl, 15 mM MgCl₂), 1 μl of dNTP mixture (10 mM of each of dATP, dGTP, dCTP and dTTP), 1 μl of 10 pmol forward primer, 1 μl of 10 pmol reverse primer, 1 μl of 1/10-diluted DNA template (100 ng) and 0.2 μl of Taq polymerase (5 units/μl, Solgent Co., Korea) was added with distilled water to a final volume of 50 μl. PCR was carried out under the following conditions: first denaturation at 94° C. for 5 minutes, 10 cycles of second denaturation at 94° C. for 50 seconds, annealing at 56° C. for 50 seconds and extension at 72° C. for 70 seconds, and 20 cycles of third denaturation at 94° C. for 50 seconds, annealing at 58° C. for 50 seconds and extension at 72° C. for 70 seconds, followed by one final extension at 72° C. for 5 minutes. The PCR products were analyzed by agarose gel electrophoresis. The analysis showed that double-stranded DNA for each strain was synthesized.

Amplified DNA products were purified using PCR purification kit (Qiagen, Co.) to add 1 μl Lamda exonuclease (New England Biolabs, Inc., Netherlands), and then allowed to react at 37° C. for 1 hour, thus obtaining single-stranded DNA. Lamda exonuclease is an enzyme degrading DNA strands having phosphate groups linked at 5′-end thereof by selective digestion. Therefore, PCR using forward primers, which have phosphate groups linked at 5′-end, results in DNA strands having phosphate groups, which is treated with Lamda exonuclease to degrade double-stranded DNA having a phosphate group attached thereto, thus resulting in the remaining single strands having fluorescent material Cy3 linked at 5′-end thereof.

### Example 5

### Hybridization and Wash

To confirm the specificity and sensitivity of the candidate probes, hybridization was performed by applying the PCR products prepared in the above Example 4 to the DNA chip prepared in the above Example 3 on which the candidate probes were immobilized. If a candidate probe showed positive hybridization signals for the species thereof, then it was additionally tested for cross-reactions (specificity) with genomic DNAs from the above 58 species.

The DNA chip was hydrated with a water vapor and then soaked in 70% ethanol to remove any probes which had not yet been immobilized on a glass slide of the DNA chip. During a hybridization reaction, fluorescence would incur the augmentation of a hybridization signal by attaching to aldehyde groups on the glass slide surface and consequently diminish the hybridization signal with the specific probe immobilized on the chip. To prevent any reduction in a hybridization signal, the DNA chip was transferred to a blocking solution (1.3 g NaBH₄, 375 ml PBS, 125 ml 100% ethanol) and then shaken for 5 minutes. The DNA chip was washed with 0.2% SDS for 5 minutes and then five times with a sterile water for 1 minute each. The DNA chip was centrifuged at 1,500 rpm for 3 minutes to remove water on the glass slide.

50 μl of the DNA fragments amplified in Example 4 was mixed with 6×SSPE hybridization buffer solution (20×SSPE: 3 M NaCl, 0.2M NaH₂PO₄—H₂0, 0.02 M EDTA, pH 7.4; 20% (v/v) formamide, Sigma Co., St. Louis, Mo.) to a final volume of 200 μl. The resulting mixture solution was applied on a glass slide onto which the probes were immobilized and covered with a probe-clip press-seal incubation chamber (Sigma Co., St. Louis, Mo.).

The hybridization chamber was added with wet tissues to prevent dehydration around the glass slide during hybridization, and then allowed to react for more than 8 hours in a static incubator at 30° C. to induce complementary binding. After the completion of hybridization, the slides were washed with 3×SSPE (0.45 M NaCl, 15 mM C₆HsNa₃0₇, pH 7.0) and then 1×SSPE (0.15 M NaCl, 5 mM C₆HsNa₃O₇, pH 7.0) for 5 minutes, respectively. The remaining moisture on the glass surface was eliminated by centrifugation (at 15,000 rpm for 3 min).

### Example 6

### Detection of Hybrids

The hybrids were detected using Arrayworks Micro Array Scanncer (ArrayWorks, Applied Precision, Inc., USA). The hybridization results are set forth in Table 3.

TABLE 3

| Probe | SEQ ID NO. | Location | Specificity |
|---|---|---|---|
| Eco001 | 1 | 23S | Not cross-reacted with any target DNA originated from |
| | | | strains in Table 2 |
| Eco001m | 2 | 23S | Not cross-reacted with any target DNA originated from 56 |
| | | | strains in Table 2 excluding S. enterica and S. sonnei, |
| | | | S. typhimurium |
| Eco001-20 | 3 | 23S | Not cross-reacted with any target DNA originated from 38 |
| | | | strains in Table 2 excluding B. cepacia, B, vulgatus, C. freundii, |
| | | | C. hominis, C. ramosum, E. aerogenes, E. cloacea, |
| | | | K. pneumoniae, M. morganii, O. anthropi, P. aeruginosa, P. mirabilis, |
| | | | Rothia, S. agalactiae, S. enerica, S. flexneri, S. marcescens, |
| | | | S. sonnei, S. typhimurium, S. wadsworthi, and V. cholera |
| Eco001-25 | 4 | 23S | Not cross-reacted with any target DNA originated from 57 |
| | | | strains in Table 2 excluding P. aeruginosa and P. mirabilis |
| Eco002 | 5 | 23S | Not cross-reacted with any target DNA originated from |
| | | | strains in Table 2 |
| Eco003-20 | 6 | 23S | Not cross-reacted with any target DNA originated from 55 |
| | | | strains in Table 2 excluding K. pneumoniae, M. morganii, S. enterica |
| | | | and S. typhimurium |
| Eco003-25 | 7 | | Not cross-reacted with any target DNA originated from 53 |
| | | | strains in Table 2 excluding C. freundii, E. cloacea, K. pneumoniae, |
| | | | M. morganii, S. enterica and S. typhimurim |

### Example 7

### Blind Test

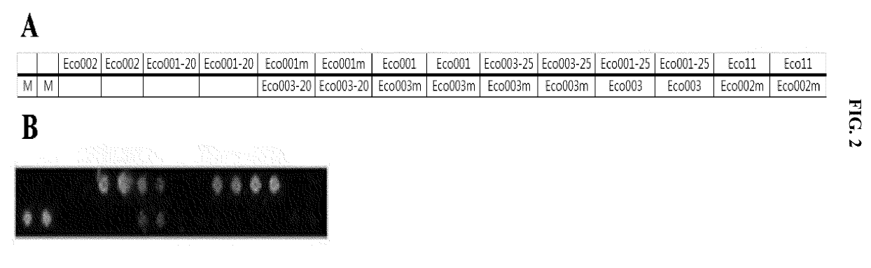

A blind test on E. coli was performed with the DNA chip constructed in Example 3. The DNA chips designed for the blind test are shown in FIG. 1A and FIG. 2A, in which marks refer to probes listed in the above Table 1. The mark “M” refers to a position marker which corresponds to the following sequence: Amine 3′-AAAAAAAAAAAAAAA-5′-FITC. The blank refers to a negative control which corresponds to a buffer (3×SSC) in which probe is dissolved.

Sixty three patients infected with pathogens were enrolled in the blind test. The samples were obtained by culturing for 1 day. The infection of samples collected from patients was confirmed by a culture method.

Genomic DNAs were isolated from cultured samples as follows. For body fluid sample, 10 ml of body fluid was collected in EDTA tube or plain tube. When the amount of sample was more than 10 ml, it was centrifuged at 5,000 rpm for 15 minutes. When the amount of sample was less than 10 ml, it was centrifuged at 14,000 rpm for 15 minutes and the precipitates formed thereby were collected in a 1.5 ml tube. The body fluid sample was suspended in 180 μl of lysozyme solution (20 mM Tris-C1, pH 8.0, 2 mM EDTA, 1.2% TritonX-100, 20 mg/ml lysozyme). The resulting suspension was cultured at 37 r for 30 minutes.

The culture was gently mixed with 20 μl of Proteinase K and 200 μl of AL solution (lysis solution, QIAamp DNA Blood Mini Kit, QIAGEN). The mixture was cultured at 55° C. for 2 hours and then at 95° C. for 10 minutes. The culture was mixed with 200 μl of 100% ethanol.

The resulting solution was loaded onto the QIAamp spin column sitting in a 2 ml tube and centrifuged at 8,000 rpm for 1 minute. The solution collected in the tube was discarded. 500 μl of AW1 solution (Wash Solution 1, QIAamp DNA Blood Mini Kit, Qiagen) was pipetted into the column which was then centrifuged at 8,000 rpm for 1 minute. The elute was discarded and 500 μl of AW2 solution (Wash Solution 2, QIAamp DNA Blood Mini Kit, Qiagen) was again pipetted into the column which was then centrifuged at 14,000 rpm for 1 minute. The elute was discarded and the QiAamp spin column was transferred to a 1.5 ml tube.

300 μl of AE solution (elution solution, DNA Blood Mini Kit, QIAGEN) was placed in the tube and stood at room temperature for 15 minutes, and then centrifuged at 8,000 rpm for 3 minutes. The eluted genomic DNAs were mixed with 750 μl of 100% ethanol and stood at −20° C. for 1 hour. The mixture was centrifuged at 14,000 rpm for 20 minutes. The ethanolic supernatant was discarded and the residue was dried. The pellet obtained thereby was dissolved in 20 μl of sterile distilled water and concentrated.

For blood sample, 10 ml of blood was placed in EDTA tube and centrifuged at 1,800 rpm at 4° C. for 10 minutes. The plasma layer was transferred to a 1.5 ml tube and centrifuged at 14,000 rpm for 10 minutes. The resulting precipitate was transferred to a 1.5 ml tube, and ensuing steps of isolating genomic DNA were the same as described above.

The procedures for amplification, hybridization, washing, and hybrid detection were performed in accordance with the same manners as described in the above Examples 4 and 5. The results are shown in Table 4 below in which the denominator is the number of sample application and the numerator is the number of hybridization signal occurred.

TABLE 4

| | Blood | Cerebral spinal fluid | Pus | Sputum | Feces | Urine |
|---|---|---|---|---|---|---|
| E. coli | 9/9 | 3/3 | 3/3 | 19/19 | 1/1 | 28/28 |

FIG. 1B and FIG. 2B show the results of hybridization on the DNA chips of FIG. 1A and FIG. 2B in blind samples including E. coli, assayed using Scanarray 5000, respectively.

### Example 8

### Effect of Antibiotic Addition on the Sensitivity of DNA Chips

The effect of antibiotic addition on the sensitivity of DNA chips constructed in Example 3, was examined by using antibiotic-treated samples.

For the strain, E. coli (ATCC 25922) was used, and for the antibiotic, Ceftriaxone (CJ, Seoul, Korea), to which E. coli has not developed resistance, was used for the sensitivity test.

First, each strain activated by two subcultures was used to estimate the antibiotic sensitivity by MIC (minimal inhibitory concentration) test. For the MIC test, a standard dilution method (Performance Standards for Antimicrobial Susceptibility Testing; Fifteenth Informational Supplement, Clinical and Laboratory Standard Institute, M100-S15, Vol. 25, No. 1, 2005) was used according to CLIS (The Clinical and Laboratory Standards Institute) guidelines. The MIC value of E. coli was 0.120 μg/ml.

E. coli diluted in BHI (Becton Dckinson, USA) was inoculated at 20˜200 CFU/ml in two flasks containing 100 ml BHI medium. Antibiotic was diluted with Endotoxin-free sterile saline (Choowae Pharma, Corp., Korea) and added to the media prepared above at 0.5 MIC, 1 MIC and 2 MIC corresponding to the concentration of each strain. For instance, 2 MIC for E. coli corresponds to 0.240 μg/ml of antibiotic addition, 1 MIC and 0.5 MIC are equivalent to the amount of 2 fold-diluted 2 MIC and 1 MIC, respectively.

After antibiotic treatment, each flask containing E. coli was shaken and incubated at 37° C. and samples were collected six times at 0 hr, 2 hrs, 4 hrs, 6 hrs, 12 hrs and 24 hrs during the culture. The sample collection was performed by pipetting 15 ml of sample from each flask and putting it into a 15 ml falcon tube. And 5 ml of each pipetted sample was placed onto BACTEC/Alert medium (Biomeriuex Inc., USA) and stationary-cultured in an Automated Blood Culture System (Biomerieux Inc., USA) maintaining a constant temperature of 35° C. Colonies obtained from the culture were subjected to additional biochemical tests for bacterial identification. 5 ml of the sample from the rest 10 ml of sample was transferred into 45 ml of BHI medium, incubated at 37° C. in a shaking incubator at 250 rpm. After 6 hours, all bacteria were collected and their genomic DNA was extracted. The final 5 ml of the sample was used directly to extract genomic DNA. Genomic DNA extraction was performed as follows; the culture broth extracted at various time points was put into 50 ml tube to centrifuge at 12,000 rpm at 4° C. for 10 minutes and the supernatant after centrifugation was discarded to obtain pellets, thus obtaining DNA using Wizard Genomic DNA Purification Kit (Promega, Co). Meanwhile, in order to estimate the number of bacteria at each time point of sample collection, 100 μl of the sample was collected and 50 μl of which was directly spread on one plate and if necessary, the rest was spread on the other plate after a 10-fold dilution or a 100-fold dilution, according to the plate count method, followed by culturing at 37° C. in an incubator.

After collecting samples, genomic DNA directly extracted from the sample and DNA collected after 6 hr of culture were amplified by PCR method described in Example 4 and the resulting PCR products were applied on DNA chips. And the result was analyzed in comparison with those of bacterial identification obtained by the culture method with Automated Blood Culture System and biochemical examinations.

Table 5 shows the results of experiments performed three times. In the table, ‘Y’ indicates identified, ‘N’ unidentified and ‘NT’ non-tested. And ‘EC’ indicates E. coli, for example, ‘EC-0.5-02’ denotes that a medium containing E. coli was treated with 0.5 MIC of an antibiotic and then harvested after 2 hr of culture. ‘Chip’ written at the upper part of table shows the results after applying samples to DNA chips and ‘Cxchip’ column shows the results of applying 6-hr cultured samples after sample collection, to DNA chips. And, ‘CX’ shows the results of bacterial identification using biochemical tests after the culture with Automated Blood Culture System.

TABLE 5
Efficacy of Detection methods depending on the concentration and
treating duration of antibiotic

| | 1^{st} experiment | | | | 2^{nd} experiment | | | | 3^{rd} experiment | | | |
|---|---|---|---|---|---|---|---|---|---|---|---|---|
| | chip | Cxchip | Cx | CFU | chip | Cxchip | Cx | CFU | chip | Cxchip | Cx | CFU |
| Start | | | | 1610 | | | | 234 | | | | |
| EC-0.5-00 | Y | Y | Y | 900 | Y | Y | Y | 130 | Y | Y | Y | 90 |
| EC-1-00 | Y | Y | Y | 900 | Y | Y | Y | 90 | N | Y | Y | 230 |
| EC-2-00 | Y | Y | Y | 1600 | Y | Y | Y | 170 | N | Y | Y | 250 |
| EC-0.5-02 | Y | N | Y | 100 | Y | Y | Y | 100 | Y | Y | Y | 10 |
| EC-1-02 | Y | Y | Y | 100 | Y | Y | Y | 0 | Y | Y | Y | 0 |
| EC-2-02 | Y | Y | Y | 0 | Y | Y | Y | 0 | Y | Y | Y | 0 |
| EC-0.5-04 | Y | Y | Y | 100 | Y | Y | Y | 0 | Y | N | Y | 0 |
| EC-1-04 | Y | Y | Y | 0 | Y | Y | N | 0 | Y | N | N | 0 |
| EC-2-04 | Y | Y | N | 0 | Y | N | N | 0 | Y | Y | N | 0 |
| EC-0.5-06 | Y | NT | Y | 400 | Y | Y | Y | 0 | Y | Y | Y | 0 |
| EC-1-06 | N | Y | N | 0 | Y | N | N | 0 | Y | Y | N | 0 |
| EC-2-06 | Y | Y | Y | 0 | Y | Y | N | 0 | Y | Y | N | 0 |
| EC-0.5-12 | Y | Y | Y | 10700 | Y | Y | N | 0 | Y | Y | Y | 110 |
| EC-1-12 | Y | Y | N | 0 | Y | Y | N | 0 | Y | N | N | 0 |
| EC-2-12 | Y | Y | N | 0 | Y | Y | N | 0 | N | N | N | 0 |
| EC-0.5-24 | N | N | Y | 4.44E+11 | Y | Y | N | 0 | Y | N | Y | 7.59E+04 |
| EC-1-24 | Y | Y | N | 0 | Y | Y | N | 0 | Y | Y | N | 0 |
| EC-2-24 | Y | Y | N | 0 | Y | Y | N | 0 | Y | Y | N | 0 |

The above experiments confirmed that the DNA chip comprising probes selected in the present invention is less sensitive to antibiotic treatment compared with the conventional culture test results, and can produce more accurate bacteria identification results.

Although the present invention has been described in detail with reference to the specific features, it will be apparent to those skilled in the art that this description is solely for a preferred embodiment and does not limit the scope of the present invention. Thus, the substantial scope of the present invention will be defined by the appended claims and equivalents thereof. It is to be appreciated that those skilled in the art can change or modify the embodiments without departing from the scope and spirit of the present invention.

### INDUSTRIAL APPLICABILITY

As described above in detail, the present invention has an effect of providing a DNA chip for detecting and identifying E. coli, on which oligonucleotides derived from 23S rRNA gene of E. coli are immobilized, and nucleic acid probes for detecting and identifying E. coli, which comprises said oligonucleotides.

The application of the DNA chip according to the present invention allows time-saving and accurate diagnosis of bacterial infection compared with the conventional of bacterial culture methods. In addition, it is not affected by antibiotics addition in clinical practice, thus leading to more accurate diagnosis.

What is claimed is:

1. Probes for detecting and identifying E. coli, which comprise one or more oligonucleotides selected from the group consisting of oligonucleotides having nucleotide sequences of SEQ ID NOs: 1˜7.

2. A DNA chip for detecting and identifying E. coli, wherein the probes of claim 1 are fixed on a substrate.

3. The DNA chip for detecting and identifying E. coli according to claim 2, wherein all oligonucleotides having nucleotide sequences of SEQ ID NOs: 1˜7 are fixed on the substrate.
